(19) United States
(12) Patent Application Publication
(10) Pub. No.: US 2017/0191026 A1
(43) Pub. Date: Jul. 6, 2017

(54) CELL CULTURE SUBSTRATE

(71) Applicant: THE UNIVERSITY OF NOTTINGHAM, Nottingham Nottinghamshire (GB)

(72) Inventors: Morgan Russell ALEXANDER, Nottingham (GB); Chris DENNING, Nottingham (GB); Lorraine YOUNG, Nottingham (GB); Adam CELIZ, Nottingham (GB); James SMITH, Nottingham (GB); Martyn Christopher DAVIES, Nottingham (GB); Asha PATEL, Nottingham (GB)

(21) Appl. No.: 15/510,556

(22) PCT Filed: Sep. 11, 2015
(86) PCT No.: PCT/GB2015/052641

§ 371 Date: Mar. 13, 2017

(30) Foreign Application Priority Data
Sep. 11, 2014 (GB) 1416078.2

(51) Int. Cl. C12N 5/00; C12M 1/34; C12M 1/00; C08L 33/26; C08F 20/18; C09D 133/26; C09D 133/08; C09D 5/03; C08F 20/58; C12N 5/074; C08L 33/08

(52) U.S. Cl. CPC C12N 5/0068; C12N 5/0696; C12M 41/12; C12M 47/02; C08L 33/26; C08L 33/08; C09D 133/26; C09D 133/08; C09D 5/03; C08F 20/58; C08F 20/18; C12N 2533/30; C12N 2523/00; C08L 2205/025; C08F 2800/20; C08L 2203/02; C08L 2205/03

(57) ABSTRACT

The invention provides a substrate for cell culture comprising a polymer, wherein the polymer comprises —a homopolymer formed from a monomer of formula (Ia) or (Ib); —a copolymer formed from one or more monomers of formula (Ia) and/or (Ib); or —a copolymer formed from one or more monomers of formula (Ia) and/or (Ib), and comprising HEMA; wherein formula (Ia) comprises: (Formula (Ia)) wherein R1 is a C8-C12 straight or branched chain alkyl or alkenyl group, for example a C8-C10 straight or branched chain alkyl group, which may optionally be substituted; and R2 is selected from H and C1-4 alkyl; and wherein formula (Ib) comprises: (Formula (Ib)) wherein R3 is a 6-12 membered ring, for example a 6-8 membered ring, which is a cycloalkyl, cycloheteroalkyl, aryl or heteroaryl group, and which may optionally be substituted; L is a divalent linker group selected from —NH—, —CH2—, and —O—; R4 is a C1-8 organic group, for example a C1-C6 organic group, wherein this group comprises at least one moiety selected from C═O, NH, NH2, COOH, and C═C.

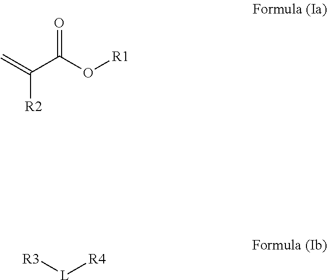

This invention relates to a substrate for culturing cells, such as human pluripotent stem cells (hPSCs); polymers, devices and methods for cell culture; polymers; and methods of manufacturing devices for cell culture.

Human pluripotent stem cells (hPSCs) hold promise towards application in regenerative medicine and drug development as well as providing new in vitro models of genetic disease due to their long-term self-renewal and inherent ability to differentiate into all three germ layers. However, for these applications to be realised, scalable culture systems capable of generating the necessary numbers of cells are needed. Currently, the most widely used growth substrates for hPSC culture are mouse embryonic fibroblast (MEF) feeder layers or Matrigel™, which is an undefined extracellular matrix (ECM) protein mixture harvested from Engelbreth-Holm-Swarm mouse sarcoma cells. To improve scalability of growth substrates and avoid the problems associated with complex biologically derived material, fully synthetic polymer-based growth substrates have been are under investigation [11-3, 16-20]. However, commercial synthetic alternatives to Matrigel™ such as Synthemax™, a peptide-acrylate conjugate coating containing a cell adhesion motif derived from vitronectin, have not proved cost-effective for large scale hPSC culture. A number of groups are therefore engaged in the search for scalable and cost effective synthetic polymer substrates on which to expand stem cells. [2-3,14,16-20].

The success of polymer-based growth substrates is critical upon the preconditioning of the substrate with various ECM proteins. This preconditioning step increases the cost of using polymeric substrates.

An aim of the present invention is to provide an improved substrate and methods for cell culture.

According to a first aspect of the invention, there is provided a substrate for cell culture comprising a polymer, wherein the polymer comprises
a homopolymer formed from a monomer of formula (Ia) or (Ib);
a copolymer formed from one or more monomers of formula (Ia) and/or (Ib); or
a copolymer formed from one or more monomers of formula (Ia) and/or (Ib), and comprising HEMA;
wherein formula (Ia) comprises:

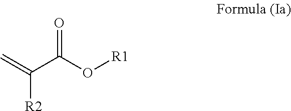

wherein

R1 is a C8-C12 straight or branched chain alkyl or alkenyl group, for example a C8-C10 straight or branched chain alkyl group, which may optionally be substituted; and R2 is selected from H and C1-4 alkyl; and
wherein formula (Ib) comprises:

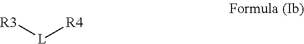

wherein

R3 is a 6-12 membered ring, for example a 6-8 membered ring, which is a cycloalkyl, cycloheteroalkyl, aryl or heteroaryl group, and which may optionally be substituted; L is a divalent linker group selected from —NH—, —CH₂—, and —O—;

R4 is a C1-8 organic group, for example a C1-C6 organic group, wherein this group comprises at least one moiety selected from C═O, NH, NH₂, COOH, and C═C.

Advantageously, the substrate of the invention provides a fully synthetic growth substrate for long-term hPSC culture in defined medium, which requires no preconditioning prior to cell culture. This polymeric material is amenable to scale up for automated hPSC expansion to achieve large numbers of cells that are necessary for clinical applications.

In an embodiment R2 may be selected from H and C1 or C2 alkyl, for example, it may be H or C1 alkyl.

The R1 group may be substituted. Thus, one or more (e.g. two or more) of the hydrogen atoms in the alkyl or alkenyl chain are replaced with substituent groups. In one embodiment from 1 to 10 hydrogen atoms in the group are substituted, such as from 1 to 6, e.g. 1, 2, 3 or 4 of the hydrogen atoms in the hydrocarbon chain might be replaced with substituent groups. When more than one hydrogen atom in the group is replaced, the substituent groups used may be the same or may be different. For example, the alkyl or alkenyl group may optionally be substituted with one or more substituent groups independently selected from fluoro, chloro, hydroxyl, amino and carboxyl groups. It may be that the alkyl or alkenyl group is optionally substituted with one or more substituent groups independently selected from fluoro and hydroxyl groups.

When R3 is a cycloheteroalkyl, or heteroaryl group, the heteroatoms in the ring may, for example, be selected from O, N, S, SO₂, P, B, Si, and combinations thereof. For example, the heteroatoms may be selected from O, N, S, and combinations thereof. In one embodiment there are from 1 to 4 heteroatoms in the ring, for example there may be 1, 2 or 3 heteroatoms in the ring.

In one embodiment, R3 is cycloalkyl or aryl, however, and thus there are no heteroatoms in the ring.

In one preferred embodiment R3 is a 6 membered ring. It may, for example, be a 6-membered cycloalkyl or a 6-membered aryl.

In one embodiment, R3 is phenyl, which may optionally be substituted with one or more substituent groups.

The R3 cyclic group may be substituted. Thus one or more (e.g. two or more) of the groups in the ring may be provided with substituent groups—i.e. one or more (e.g. two or more) of the hydrogen atoms are substituted. In one embodiment from 1 to 10 hydrogen atoms in the group are substituted, such as from 1 to 6. For example 1, 2, 3 or 4 of the hydrogen atoms in the group might be replaced with substituent groups, e.g. 1 or 2. When more than one hydrogen atom in the group is replaced, the substituent groups used may be the same or may be different.

For example, the cyclic group may optionally be substituted with one or more substituent groups independently selected from fluoro, chloro, hydroxyl, amino and carboxyl groups. It may be that the cyclic group is optionally substituted with one or more substituent groups independently selected from fluoro and hydroxyl groups. In one preferred embodiment any subsistent groups present are hydroxyl groups. For example, there may be zero, one or two hydroxyl subsistuent groups on the ring.

The R4 group may be straight chain or may be branched.

In one embodiment, the R4 group is a C1-C5 organic group, such as a C1-C4 organic group.

It may be that the R4 organic group comprises two or more moieties selected from C═O, NH, NH₂, COOH, and C═C. For example, it may comprise a C═O and a C═C moiety, or it may comprise NH and COOH. R4 may further comprise a CH3 moiety.

In one embodiment, the R4 group is

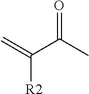

where R2 is selected from H and C1-4 alkyl; preferably it is selected from H and C1 or C2 alkyl, for example it may be H or C1 alkyl.

The monomer of formula (Ia) may comprise any of the monomers selected from:
2-Ethylhexyl methacrylate:

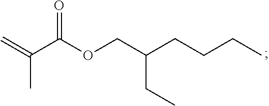

Octafluoro-2-hydroxy-6-(trifluoromethyl)heptyl methacrylate:

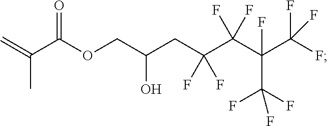 [SMILES: C=C(C)C(=O)OCC(O)CC(F)(F)C(F)(F)C(F)(C(F)(F)F)C(F)(F)F]

Dodecafluoroheptyl acrylate:

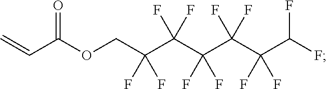 [SMILES: C=CC(=O)OCC(F)(F)C(F)(F)C(F)(F)C(F)(F)C(F)(F)C(F)F]

or
Lauryl methacrylate:

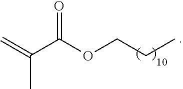

The monomer of formula (Ib) may comprise any of the monomers selected from:
2-Norbornyl methacrylate:

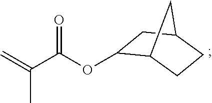

N-(4-Hydroxyphenyl)methacrylamide:

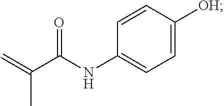

N-Phenylmethacrylamide:

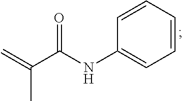

Cyclohexyl methacrylate:

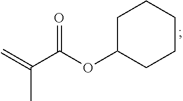

Tris[2-(acryloyloxy)ethyl] isocyanurate:

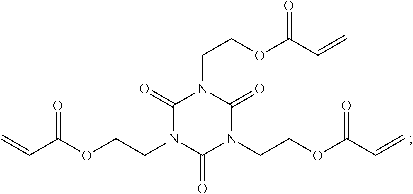 [SMILES: C=CC(=O)OCCn1c(=O)n(CCOC(=O)C=C)c(=O)n(CCOC(=O)C=C)c1=O]

Poly-L-Tyrosine:

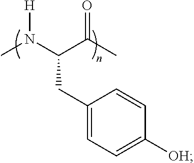

or
Dopamine:

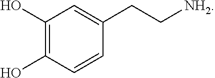 [SMILES: NCCc1ccc(O)c(O)c1]

The monomer of formula (Ib) may comprise any of the monomers selected from 2-Norbornyl methacrylate; N-(4-Hydroxyphenyl)methacrylamide; N-Phenylmethacrylamide; Cyclohexyl methacrylate; or Tris[2-(acryloyloxy)ethyl] isocyanurate.

The monomer of formula (Ib) may comprise Poly-L-Tyrosine. The monomer of formula (Ib) may comprise Dopamine. Poly-L-Tyrosine may be provided in a copolymer with one or more other monomers of formula (Ia) and/or (Ib), and optionally HEMA. Dopamine may be provided in a copolymer with one or more other monomers of formula (Ia) and/or (Ib), and optionally HEMA.

The substrate of the invention advantageously provides a substrate for long-term hPSC culture in defined medium, which requires no preconditioning prior to cell culture. This polymeric material is amenable to scale up for automated hPSC expansion to achieve large numbers of cells that are necessary for clinical applications.

It is understood that hydroxyethyl methacrylate (HEMA), also known as poly(2-hydroxyethyl methacrylate), is a monomer which polymerises to form of PolyHEMA, or pHEMA.

The substrate may comprise a polymer blend of two or more different polymers. The substrate may comprise a polymer blend of three or more different polymers. The substrate may comprise a polymer blend of two or three different polymers. Second and/or third polymers may comprise a homopolymer formed from a monomer of formula (Ia) or (Ib); or a copolymer formed from one or more monomers of formula (Ia) and/or I(b); or a copolymer formed from one or more monomers of formula (Ia) and/or (Ib) and comprising HEMA.

The substrate polymer may be a first polymer, and the substrate may further comprise a different second polymer, wherein the second polymer may comprise:
a homopolymer formed from a monomer of formula (Ia) or (Ib);
a copolymer formed from one or more monomers of formula (Ia) and/or (Ib); or
a copolymer formed from one or more monomers of formula (Ia) and/or (Ib) and comprising HEMA.

Second, third or subsequent polymers may be blended with the polymer such that they are intermixed. Alternatively, the substrate may comprise second, third or subsequent polymers that are separated into distinct regions relative to the first polymer. Second, third or subsequent polymers may not be blended with the first polymer (e.g not intermixed). Distinct regions of polymers may comprise arrangements of strips, spots, lattices, or layers of one type of polymer alongside distinct regions or layers of another polymer. The polymer may be arranged on the substrate in patterns, for example, to influence cell adhesion patterns. The patterns may be arranged to provide a pre-determined tissue architecture

The regions of polymers may be arranged to recreate natural cell spacing and tissue architecture in vitro. The regions of polymers may be arranged to align cells. For example, alignment of cardiomyocytes can be provided such that they contract in one direction similar to heart cells in vivo, thereby the in vitro heart tissue may generate optimal force and produce cells of optimal maturity.

Neighbouring regions of polymers may have different properties and can be printed or coated in a particular pattern to promote adhesion and differentiation. For example, hepatic differentiation and polarisation of the cells may be promoted to form primary cells to function as a model liver tissue. Spots of the substrate on the cell culture device service may promote more efficient colony growth relative to uniform substrate coverage.

The polymer may comprise or consist of N-(4-Hydroxyphenyl)methacrylamide or N-Phenylmethacrylamide, or a combination thereof. In one embodiment of the invention the monomer may comprise or consist of N-(4-Hydroxyphenyl)methacrylamide. In alternative embodiment the monomer may comprise or consist of N-Phenylmethacrylamide.

The copolymer may comprise two or more monomers selected from the group comprising 2-Norbornyl methacrylate; 2-Ethylhexyl methacrylate; N-(4-Hydroxyphenyl)methacrylamide; Octafluoro-2-hydroxy-6-(trifluoromethyl)heptyl methacrylate; Lauryl methacrylate; N-Phenylmethacrylamide; Cyclohexyl methacrylate; Dodecafluoroheptyl acrylate; and Tris[2-(acryloyloxy)ethyl] isocyanurate.

The copolymer may comprise 2-Norbornyl methacrylate and a monomer selected from the group comprising 2-Ethylhexyl methacrylate; N-(4-Hydroxyphenyl)methacrylamide; Octafluoro-2-hydroxy-6-(trifluoromethyl)heptyl methacrylate; Lauryl methacrylate; N-Phenylmethacrylamide; Cyclohexyl methacrylate; Dodecafluoroheptyl acrylate; and Tris[2-(acryloyloxy)ethyl] isocyanurate.

The copolymer may comprise 2-Ethylhexyl methacrylate and a monomer selected from the group comprising 2-Norbornyl methacrylate; N-(4-Hydroxyphenyl)methacrylamide; Octafluoro-2-hydroxy-6-(trifluoromethyl)heptyl methacrylate; Lauryl methacrylate; N-Phenylmethacrylamide; Cyclohexyl methacrylate; Dodecafluoroheptyl acrylate; and Tris[2-(acryloyloxy)ethyl] isocyanurate.

The copolymer may comprise N-(4-Hydroxyphenyl)methacrylamide and a monomer selected from the group comprising 2-Norbornyl methacrylate; 2-Ethylhexyl methacrylate; Octafluoro-2-hydroxy-6-(trifluoromethyl)heptyl methacrylate; Lauryl methacrylate; N-Phenylmethacrylamide; Cyclohexyl methacrylate; Dodecafluoroheptyl acrylate; and Tris[2-(acryloyloxy)ethyl] isocyanurate.

The copolymer may comprise Octafluoro-2-hydroxy-6-(trifluoromethyl)heptyl methacrylate and a monomer selected from the group comprising 2-Norbornyl methacrylate; 2-Ethylhexyl methacrylate; N-(4-Hydroxyphenyl)methacrylamide; Lauryl methacrylate; N-Phenylmethacrylamide; Cyclohexyl methacrylate; Dodecafluoroheptyl acrylate; and Tris[2-(acryloyloxy)ethyl] isocyanurate.

The copolymer may comprise Lauryl methacrylate and a monomer selected from the group comprising 2-Norbornyl methacrylate; 2-Ethylhexyl methacrylate; N-(4-Hydroxyphenyl)methacrylamide; Octafluoro-2-hydroxy-6-(trifluoromethyl)heptyl methacrylate; Lauryl methacrylate; N-Phenylmethacrylamide; Dodecafluoroheptyl acrylate; and Tris[2-(acryloyloxy)ethyl] isocyanurate.

The copolymer may comprise N-Phenylmethacrylamide and a monomer selected from the group comprising 2-Norbornyl methacrylate; 2-Ethylhexyl methacrylate; N-(4-Hydroxyphenyl)methacrylamide; Octafluoro-2-hydroxy-6-(trifluoromethyl)heptyl methacrylate; Lauryl methacrylate; Cyclohexyl methacrylate; Dodecafluoroheptyl acrylate; and Tris[2-(acryloyloxy)ethyl] isocyanurate.

The copolymer may comprise Cyclohexyl methacrylate and a monomer selected from the group comprising 2-Norbornyl methacrylate; 2-Ethylhexyl methacrylate; N-(4-Hydroxyphenyl)methacrylamide; Octafluoro-2-hydroxy-6-(trifluoromethyl)heptyl methacrylate; Lauryl methacrylate; N-Phenylmethacrylamide; Dodecafluoroheptyl acrylate; and Tris[2-(acryloyloxy)ethyl] isocyanurate.

The copolymer may comprise Dodecafluoroheptyl acrylate and a monomer selected from the group comprising 2-Norbornyl methacrylate; 2-Ethylhexyl methacrylate; N-(4-Hydroxyphenyl)methacrylamide; Octafluoro-2-hydroxy-6-(trifluoromethyl)heptyl methacrylate; Lauryl methacrylate; N-Phenylmethacrylamide; Cyclohexyl methacrylate; and Tris[2-(acryloyloxy)ethyl] isocyanurate.

The copolymer may comprise Tris[2-(acryloyloxy)ethyl] isocyanurate and a monomer selected from the group comprising 2-Norbornyl methacrylate; 2-Ethylhexyl methacrylate; N-(4-Hydroxyphenyl)methacrylamide; Octafluoro-2-hydroxy-6-(trifluoromethyl)heptyl methacrylate; Lauryl methacrylate; N-Phenylmethacrylamide; Cyclohexyl methacrylate; and Dodecafluoroheptyl acrylate.

The copolymer may comprise combinations of three or more monomers selected from the group comprising 2-Norbornyl methacrylate; 2-Ethylhexyl methacrylate; N-(4-Hydroxyphenyl)methacrylamide; Octafluoro-2-hydroxy-6-(trifluoromethyl)heptyl methacrylate; Lauryl methacrylate; N-Phenylmethacrylamide; Cyclohexyl methacrylate; Dodecafluoroheptyl acrylate; and Tris[2-(acryloyloxy)ethyl] isocyanurate.

The copolymer may comprise a monomer of HEMA and one or more monomers selected from the group comprising 2-Norbornyl methacrylate; 2-Ethylhexyl methacrylate; N-(4-Hydroxyphenyl)methacrylamide; Octafluoro-2-hydroxy-6-(trifluoromethyl)heptyl methacrylate; Lauryl methacrylate; N-Phenylmethacrylamide; Cyclohexyl methacrylate; Dodecafluoroheptyl acrylate; and Tris[2-(acryloyloxy)ethyl] isocyanurate; or combinations thereof. The copolymer may comprise a monomer of HEMA and N-(4-Hydroxyphenyl)methacrylamide.

Advantageously, providing a copolymer comprising HEMA can improve the material properties of the polymer coating in well plates, for example to reduce brittleness of the polymer and avoid cracking of polymer coating.

The average molecular weight of the polymer or copolymer strands may be about 100 kDa per strand. The average molecular weight of the polymer or copolymer strands may be at least about 1 kDa per strand. The average molecular weight of the polymer or copolymer strands may be at least about 10 kDa per strand. The average molecular weight of the polymer or copolymer strands may be at least about 50 kDa per strand. The average molecular weight of the polymer or copolymer strands may be less than about 10,000 kDa per strand. The average molecular weight of the polymer or copolymer strands may be less than about 5000 kDa per strand. The average molecular weight of the polymer or copolymer strands may be less than about 1000 kDa per strand. The average molecular weight of the polymer or copolymer strands may be less than about 500 kDa per strand. The average molecular weight of the polymer or copolymer strands may be less than about 200 kDa per strand. The average molecular weight of the polymer or copolymer strands may be between about 1 kDa and about 10,000 kDa per strand. The average molecular weight of the polymer or copolymer strands may be between about 10 kDa and about 1000 kDa per strand. The average molecular weight of the polymer or copolymer strands may be between about 50 kDa and about 1000 kDa per strand. The average molecular weight of the polymer or copolymer strands may be between about 100 kDa and about 1000 kDa per strand. The average molecular weight of the polymer or copolymer strands may be between about 10 kDa and about 500 kDa per strand. The average molecular weight of the polymer or copolymer strands may be between about 10 kDa and about 200 kDa per strand. The average molecular weight of the polymer or copolymer strands may be between about 80 kDa and about 150 kDa per strand.

In an embodiment where the polymer comprises a copolymer, the copolymer may comprise at least 10% HEMA. The copolymer may comprise at least 50 wt % HEMA. The copolymer may comprise at least 60 wt % HEMA. The copolymer may comprise at least 70 wt % HEMA. The copolymer may comprise at least 80 wt % HEMA. The copolymer may comprise at least 90% HEMA.

The copolymer may comprise N-(4-Hydroxyphenyl)methacrylamide and HEMA. The copolymer may comprise N-(4-Hydroxyphenyl)methacrylamide and at least 10 wt % HEMA. The copolymer may comprise N-(4-Hydroxyphenyl)methacrylamide and at least 50 wt % HEMA. The copolymer may comprise N-(4-Hydroxyphenyl)methacrylamide and at least 60 wt % HEMA. The copolymer may comprise N-(4-Hydroxyphenyl)methacrylamide and at least 70 wt % HEMA. The copolymer may comprise N-(4-Hydroxyphenyl)methacrylamide and at least 80 wt % HEMA. The copolymer may comprise N-(4-Hydroxyphenyl)methacrylamide and at least 90% HEMA.

The copolymer may comprise N-Phenylmethacrylamide and HEMA. The copolymer may comprise N-Phenylmethacrylamide and at least 10 wt % HEMA. The copolymer may comprise N-Phenylmethacrylamide and at least 50 wt % HEMA. The copolymer may comprise N-Phenylmethacrylamide and at least 60 wt % HEMA. The copolymer may comprise N-Phenylmethacrylamide and at least 70 wt % HEMA. The copolymer may comprise N-Phenylmethacrylamide and at least 80 wt % HEMA. The copolymer may comprise N-Phenylmethacrylamide and at least 90% HEMA.

The copolymer may comprise a ratio of HEMA to monomer of formula (Ia) and/or (Ib) of at least 1:10. The copolymer may comprise a ratio of HEMA to monomer of formula (Ia) and/or (Ib) of at least 1:5. The copolymer may comprise a ratio of HEMA to monomer of formula (Ia) and/or (Ib) of at least 1:2. The copolymer may comprise a ratio of HEMA to monomer of formula (Ia) and/or (Ib) of at least 1:1. The copolymer may comprise a ratio of HEMA to monomer of formula (Ia) and/or (Ib) of at least 2:1. The copolymer may comprise a ratio of HEMA to monomer of formula (Ia) and/or (Ib) of at least 5:1. The copolymer may comprise a ratio of HEMA to monomer of formula (Ia) and/or (Ib) of at least 10:1.

The copolymer may comprise a ratio of HEMA to N-(4-Hydroxyphenyl)methacrylamide or N-Phenylmethacrylamide of at least 1:10. The copolymer may comprise a ratio of HEMA to N-(4-Hydroxyphenyl)methacrylamide or N-Phenylmethacrylamide of at least 1:5. The copolymer may comprise a ratio of HEMA to N-(4-Hydroxyphenyl)methacrylamide or N-Phenylmethacrylamide of at least 1:2. The copolymer may comprise a ratio of HEMA to N-(4-Hydroxyphenyl)methacrylamide or N-Phenylmethacrylamide of at least 1:1. The copolymer may comprise a ratio of HEMA to N-(4-Hydroxyphenyl)methacrylamide or N-Phenylmethacrylamide of at least 2:1. The copolymer may comprise a ratio of HEMA to N-(4-Hydroxyphenyl)methacrylamide or N-Phenylmethacrylamide of at least 5:1 The copolymer may comprise a ratio of HEMA to N-(4-Hydroxyphenyl)methacrylamide or N-Phenylmethacrylamide of at least 10:1.

The copolymer may comprise any one of the combinations and percentages of monomers as recited in Table 1 herein.

The polymer may be unbranched, or substantially unbranched.

The substrate for cell culture may be a coating on the surface of a cell culture device. The substrate may be for coating the surface of a cell culture device. The substrate may be layered, or may form a layer, on a cell culture device, such as a tissue culture plate.

The substrate may be in a solution, arranged to be applied to a surface of a cell culture device. The solution may comprise a solvent such as ethanol. The substrate may be in powdered form, for example, the substrate may be arranged to be dissolved in solution and applied to a cell culture device.

The cell culture device may comprise a tissue culture plate. The cell culture device may comprise a multiwell culture plate. The cell culture device may comprise 6, 24, 96 or more wells. The cell culture device may comprise a 2-D surface for cell culture, or a 3-D architecture. The 3-D architecture may be a porous matrix. The device may comprise a 3-D culture system with microspheres, wherein the substrate may be coated on the microspheres. The device may be a bioreactor, such as a multi-well bioreactor. The device may be a perfusion, or pumped media, bioreactor. The device may be a microfluidic device, for example, for drug screening of stem cells. The device may be an organ-on-chip device, wherein the substrate coating may aid tissue formation.

The cell culture device may comprise any suitable material for culturing cells. The cell culture device may comprise polystyrene. The cell culture device may comprise glass. The cell culture device may comprise polypropylene. The cell culture device may comprise polypropylene. The cell culture device may comprise polyurethane. The surface of the cell culture device may be oxygen-plasma etched.

The substrate coating on the cell culture device may comprise applying a polymer solution in a solvent to the cell culture device, followed by evaporation of the solvent to leave a polymer layer on the surface of the cell culture device. The solvent may comprise or consist of ethanol.

The chemical constituents of the substrate may be defined. The substrate may be entirely polymeric. The substrate may be entirely synthetic. The substrate may not comprise biological source material, such as material from cells. The substrate may not comprise extracellular matrix.

The term “biological source material” is understood to refer to material derived from a biological source, for example an extract from a cell, cell culture, tissue, or secretion. The cells to be cultured with or on the substrate may comprise or consist of stem cells; partially or fully differentiated cells, such as fibroblasts; or progenitor cells; or combinations thereof.

The cells may be mammalian. The cells may be human.

The cells may comprise or consist of stem cells. The cells may comprise or consist of pluripotent stem cells. The cells may comprise or consist of multipotent stem cells. The cells may comprise or consist of pluripotent stem cells, such as human pluripotent stem cells (hPSCs). The cells may comprise or consist of mesenchymal stem cells, such as human mesenchymal stem cells (hMSCs).

Advantageously, the substrate of the invention is capable of keeping cells such as hPSCs alive for a greater time and greater number of passages than cell cultures without the substrate of the invention. Pluripotency can also be maintained, which is important for research and maintaining cell banks.

The substrate may be for viable cell culture. Viable cell culture may be defined as the ability to keep cells alive over a period of time; the ability to expand the cell number; the ability to grow the cells to confluence in a culture; and or the ability to passage the cells for one or more passages.

The cell culture may be for maintaining live cells for at least 24 hours. The cell culture may be for maintaining live cells for at least 48 hours. The cell culture may be for maintaining live cells for at least 60 hours. The cell culture may be for maintaining live cells for at least 72 hours. The cell culture may be for maintaining live cells for at least 3 days. The cell culture may be for maintaining live cells for at least 4 days. The cell culture may be for maintaining live cells for at least 5 days. The cell culture may be for maintaining live cells for at least 10 days. The cell culture may be for maintaining live cells for at least 20 days. Maintaining live cells may comprise one or more passages for the period of culture.

The cell culture may be for maintaining live cells, such as hPSCs, for at least 2 passages. The cell culture may be for maintaining live cells, such as hPSCs, for at least 3 passages. The cell culture may be for maintaining live cells, such as hPSCs, for at least 4 passages. The cell culture may be for maintaining live cells, such as hPSCs, for at least 5 passages. The cell culture may be for maintaining live cells, such as hPSCs, for at least 6 passages. A passage may take place after about 5 days of culture. A passage may take place after about 7 days of culture. A passage may take place after about 2 or 3 days of culture. The cell culture may be for maintaining live cells, such as hPSCs, for at least 2 passages, with at least 72 hours between passages.

The cell culture may be for maintaining live cells, such as hPSCs, for at least 4 days. The cell culture may be for maintaining live cells, such as hPSCs, for at least 6 days. The cell culture may be for maintaining live cells, such as hPSCs, for at least 10 days. The cell culture may be for maintaining live cells, such as hPSCs, for at least 14 days. The cell culture may be for maintaining live cells, such as hPSCs, for at least 20 days.

In an embodiment where pluripotent stem cells are to be cultured, such as hPSCs, the cell culture may be for maintaining pluripotency. The cell culture may be for maintaining pluripotency for at least 12 hours. The cell culture may be for maintaining pluripotency for at least 24 hours. The cell culture may be for maintaining pluripotency for at least 48 hours. The cell culture may be for maintaining pluripotency for at least 60 hours. The cell culture may be for maintaining pluripotency for at least 72 hours. The cell culture may be for maintaining pluripotency for at least 3 days. The cell culture may be for maintaining pluripotency for at least 4 days. The cell culture may be for maintaining pluripotency for at least 5 days. The cell culture may be for maintaining pluripotency for at least 8 days. The cell culture may be for maintaining pluripotency for at least 10 days. The cell culture may be for maintaining pluripotency for at least 20 days. Maintaining pluripotent cells may comprise one or more passages for the period of culture.

The cell culture may be for maintaining pluripotency for at least 1 passage. The cell culture may be for maintaining pluripotency for at least 2 passages. The cell culture may be for maintaining pluripotency for at least 3 passages. The cell culture may be for maintaining pluripotency for at least 4 passages. The cell culture may be for maintaining pluripotency for at least 5 passages.

The passage may comprise passaging by enzyme-free dissociation. The passage may comprise mechanical passaging. The passage may comprise enzyme mediated dissociation.

Pluripotency may be readily determined by the skilled person and may include determination of all, or combinations of, the following pluripotent cell characteristics.
1) Morphology wherein the cells have a high nuclear to cytoplasmic ratio;
2) The cells undergo serial passage and maintain consistent population doubling rates;
3) The cells express markers of the stem cell state. These include genes and proteins (including but not limited to OCT4, NANOG, SOX2, TDGF, DNMT3B, REX1) and glycolipids (including but not limited to TRA181, TRA160, SSEA3, SSEA4);
4) The cells differentiate to representatives of the three germ layers (ectoderm, endoderm and mesoderm) in vitro; and optionally
5) The cells have the potential to form teratomas in immunocompromised mice and in these structures representatives of the three germ layers (ectoderm, endoderm and mesoderm) can be found.

In an embodiment where differentiated cells are to be cultured, such as fibroblasts, the cell culture may be for maintaining differentiation. The cell culture may be for maintaining differentiation for at least 12 hours. The cell culture may be for maintaining differentiation for at least 24 hours. The cell culture may be for maintaining differentiation for at least 48 hours. The cell culture may be for maintaining differentiation for at least 60 hours. The cell culture may be for maintaining differentiation for at least 72 hours. The cell culture may be for maintaining differentiation for at least 3 days. The cell culture may be for maintaining differentiation for at least 4 days. The cell culture may be for maintaining differentiation for at least 5 days. The cell culture may be for maintaining differentiation for at least 8 days. The cell culture may be for maintaining differentiation for at least 10 days. The cell culture may be for maintaining differentiation for at least 20 days. Maintaining differentiation cells may comprise one or more passages for the period of culture.

The cell culture may be for maintaining differentiation for at least 1 passage. The cell culture may be for maintaining differentiation for at least 2 passages. The cell culture may be for maintaining differentiation for at least 3 passages. The cell culture may be for maintaining differentiation for at least 4 passages. The cell culture may be for maintaining differentiation for at least 5 passages.

According to another aspect of the invention, there is provided a cell culture device comprising the substrate for culturing cells in accordance with the invention.

The device may be a cell culture plate. The device may be a tissue culture plate. The device may be a multi-well plate. The device may be a bioreactor, such as a multi-well bioreactor. The device may be a perfusion, or pumped media, bioreactor.

The substrate may form a surface region on the cell culture plate for cell adhesion, such as hPSC adhesion.

The device may be arranged to be, or capable of being, stored for at least 6 months, whilst retaining viability for culturing cells for this period. The device may be arranged to be, or capable of being, stored for at least 12 months, whilst retaining viability for culturing cells for this period. The device may be arranged to be, or capable of being, stored for at least 2 year, whilst retaining viability for culturing cells for this period.

According to another aspect of the invention, there is provided a polymer for use as a substrate for facilitating cell adhesion on a cell culture plate; wherein the polymer comprises
a homopolymer formed from a monomer of formula (Ia) or (Ib);
a copolymer formed from one or more monomers of formula (Ia) and/or (Ib); or
a copolymer formed from one or more monomers of formula (Ia) and/or I(b), and comprising HEMA.

According to another aspect of the invention, there is provided the use of the polymer according to the invention as a substrate for facilitating cell adhesion on a cell culture plate.

Without being bound by theory, the polymer may facilitate adhesion via the initial adsorption of essential ECM proteins in the correct conformation from the culture medium, these proteins can subsequently interact with cell-surface adhesion integrins to facilitate attachment to the surface.

According to another aspect of the invention, there is provided a method of culturing cells, the method comprising the steps of:

providing a device for cell culture, wherein the device for cell culture comprises a surface layered with the substrate in accordance with the invention;

introducing cell culture media and the cells; and

incubating the device at a temperature suitable for maintenance and/or growth of the cells.

The method may further comprise the step of harvesting cultured cells from the device, wherein the cells are harvested by enzymatic removal from the surface of the device.

The method may further comprise the step of harvesting cultured cells from the device, wherein the cells are not harvested by scraping the cells from the surface of the device.

Incubating the device at a temperature suitable for maintenance and/or growth of the cells may be at about 37° C. The incubation may be at about a 5% CO₂. The incubation may comprise shaking, stirring, rocking, or agitation of the cell media.

The method of culturing cells may not comprise a preconditioning step for the device for cell culture. Preconditioning may comprise treating the surface of the device with proteins, cell extracts, or extracellular matrix. Preconditioning may comprise incubating the device with media, for example for at least an hour before the cell culture is provided.

The cell culture may be for at least 24 hours. The cell culture may be at least 48 hours. The cell culture may be at least 60 hours. The cell culture may be for at least 72 hours. The cell culture may be for at least 3 days. The cell culture may be for at least 4 days.

The cell culture may be for at least 5 days. The cell culture may be for at least 10 days. The cell culture may be for at least 20 days. The method may comprise one or more passages for the period of culture. The method may comprise two or more passages for the period of culture. The method may comprise three or more passages for the period of culture. The method may comprise four or more passages for the period of culture.

The cell culture may comprise hPSCs maintained for at least 2 passages. The cell culture may comprise hPSCs maintained for at least 3 passages. The cell culture may comprise hPSCs maintained for at least 4 passages. The cell culture may comprise hPSCs maintained for at least 5 passages. The cell culture may comprise hPSCs maintained for at least 6 passages. A passage may take place after about 5 days of culture. A passage may take place after about 7 days of culture. A passage may take place after about 2 or 3 days of culture. The cell culture may be for maintaining live cells, such as hPSCs, for at least 2 passages, with at least 72 hours between passages.

The cell culture may comprise hPSCs maintained for at least 4 days. The cell culture may comprise hPSCs maintained for at least 6 days. The cell culture may comprise hPSCs maintained for at least 10 days. The cell culture may comprise hPSCs maintained for at least 14 days. The cell culture may comprise hPSCs maintained for at least 20 days.

In an embodiment where pluripotent stem cells are cultured, such as hPSCs, the cell culture may maintain pluripotency. The cell culture may maintain pluripotency for at least 12 hours. The cell culture may maintain pluripotency for at least 24 hours. The cell culture may maintain pluripotency for at least 48 hours. The cell culture may maintain pluripotency for at least 60 hours. The cell culture may maintain pluripotency for at least 72 hours. The cell culture may maintain pluripotency for at least 3 days. The cell culture may maintain pluripotency for at least 4 days. The cell culture may maintain pluripotency for at least 5 days. The cell culture may maintain pluripotency for at least 8 days. The cell culture may maintain pluripotency for at least 10 days. The cell culture may maintain pluripotency for at least 20 days.

The passage may comprise passaging by enzyme-free dissociation. The passage may comprise mechanical passaging. The passage may comprise enzyme mediated dissociation.

The cell culture media may be any media suitable for the cell type to be cultured, such as suitable for hPSC culture. The cell culture media may be any media arranged to support cell growth. The cell culture media may be any media arranged to support cell pluripotency. The cell culture media may be any media arranged to support cell differentiation. Commercially or non-commercially available cell media may be used, for example Nutristem (Stemgent Inc.), E8, Stempro or mTeSR (Life Technologies Inc.). Undefined mouse embryonic fibroblast conditioned media may also be used.

The method of culturing cells may comprise growing or maintaining stem cells, such as hPSCs, on the substrate, followed by removing the cells from the substrate and differentiating the cells. Alternatively, the method of culturing cells may comprise growing or maintaining stem cells, such as hPSCs, on the substrate, followed by differentiating the cells on the substrate.

According to another aspect of the invention, there is provided a method of maintaining pluripotency of hPSCs in culture in vitro, comprising culturing the hPSCs on a substrate in accordance with the invention herein.

According to another aspect of the invention, there is provided a method of maintaining multipotency of a MSC cell in a culture in vitro, comprising culturing the MSC cell on a substrate in accordance with the invention herein.

According to another aspect of the invention, there is provided a method of maintaining differentiation of a differentiated cell in a culture in vitro, comprising culturing the differentiated cell on a substrate in accordance with the invention herein.

According to another aspect of the invention, there is provided a method of promoting differentiation of a stem cell in a culture in vitro, comprising culturing the stem cell on a substrate in accordance with the invention herein.

According to another aspect of the invention, there is provided a method of maintaining a stem cell culture in vitro during the promotion of differentiation of the stem cells, the method comprising culturing the stem cell on a substrate in accordance with the invention herein.

The stem cell may be a non-embryonic stem cell.

According to another aspect of the invention, there is provided the use of the device according to the invention for culturing cells, optionally wherein the cells comprise human pluripotent stem cells.

According to another aspect of the invention, there is provided a method of manufacturing a cell culture plate for culturing cells, wherein the cell culture plate is provided with coating of the substrate according to the invention herein.

The substrate may be manufactured by polymer self-assembly, or copolymer self-assembly. The substrate may be manufactured by uncontrolled free radical polymerisation of polymers or copolymers. Polymerisation may be initiated by UV treatment.

The substrate may be coated on the cell culture device by printing. The printing may comprise 3D printing. The substrate may be coated on the cell culture device by applying a solution of the polymer in a solvent to the cell culture device, and allowing or causing evaporation of the solvent. The surface of the cell culture device may be oxygen-free radical etched prior to application of the substrate. The substrate may not be provided by reaction with a cross-linker. The substrate may not be provided by conjugation with a peptide.

According to another aspect of the invention, there is provided a method of manufacturing stem cells, comprising culturing stem cells in the device in accordance with the invention, or culturing cells according to the method of the invention.

Cell culture devices comprising the substrate of the invention may be prepared by the methods described in WO 2004043588 A2, which is herein incorporated by reference.

The skilled person will understand that optional features of one embodiment or aspect of the invention may be applicable, where appropriate, to other embodiments or aspects of the invention.

Embodiments of the invention will now be described in more detail, by way of example only, with reference to the accompanying drawings.

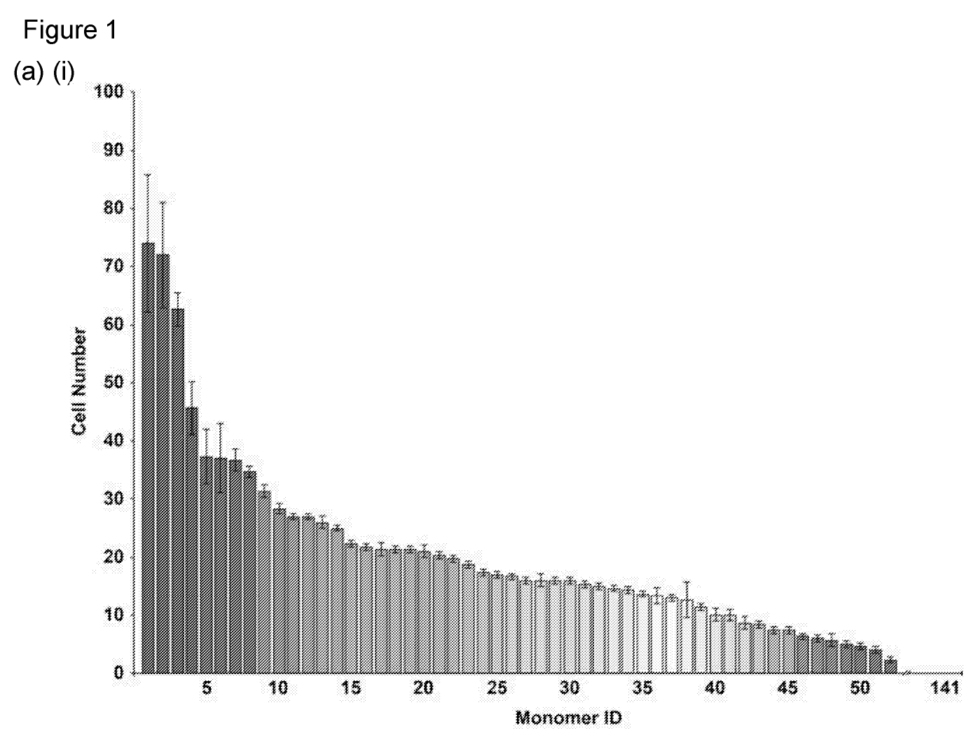

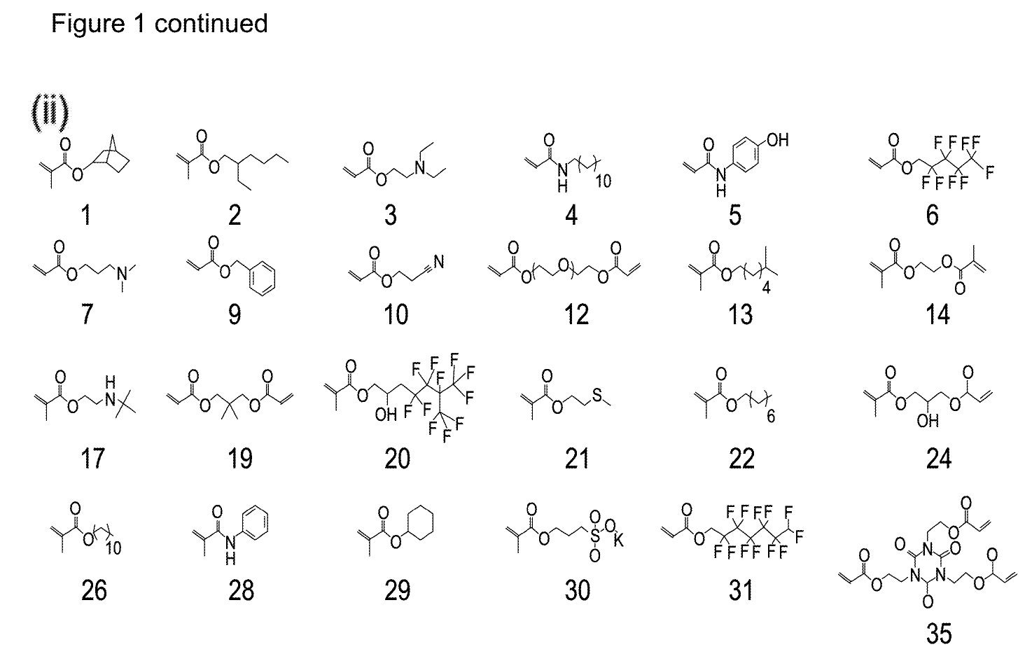

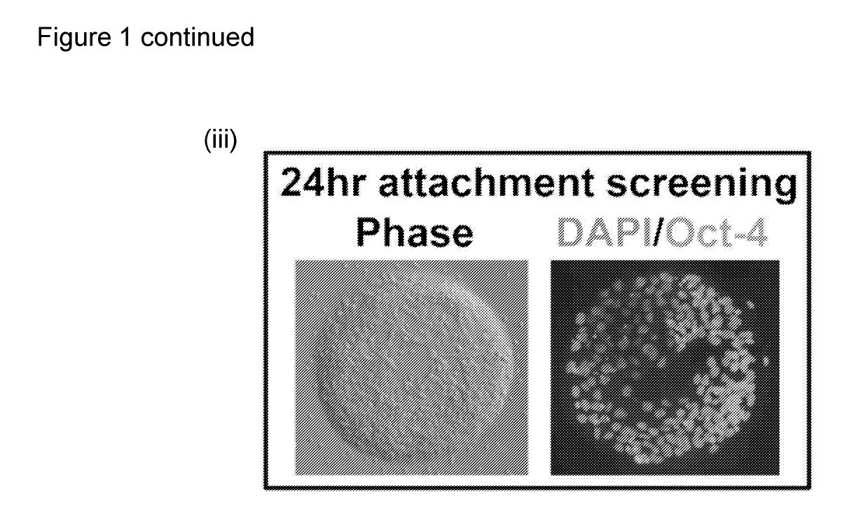

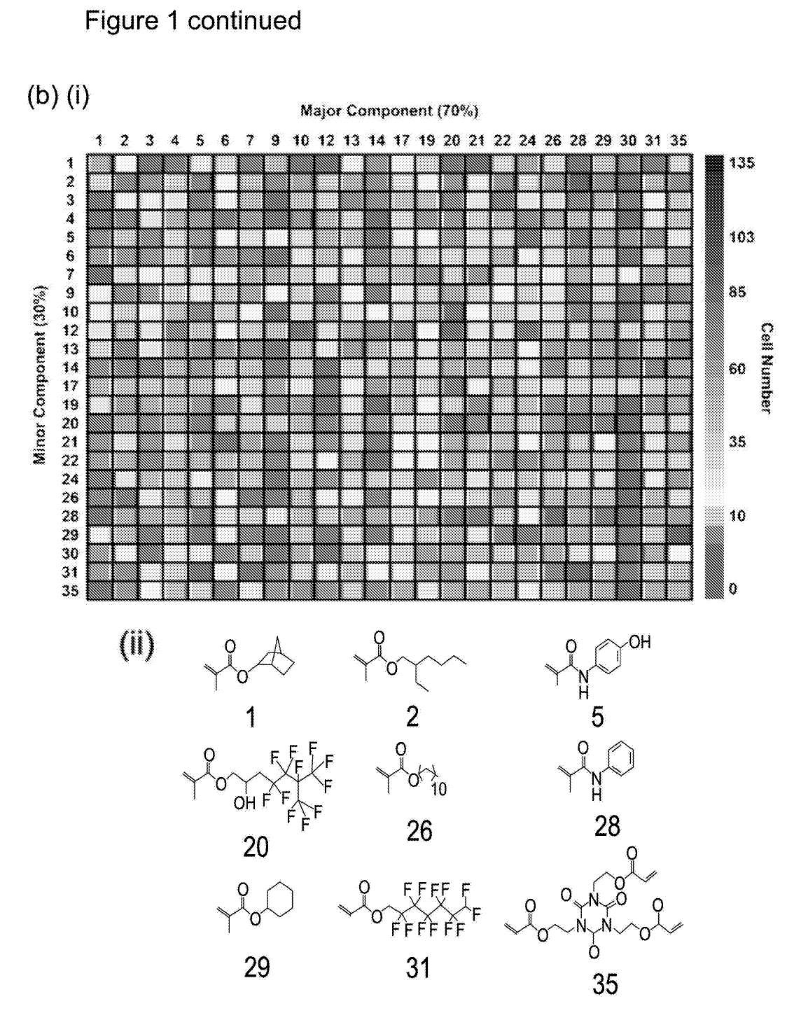

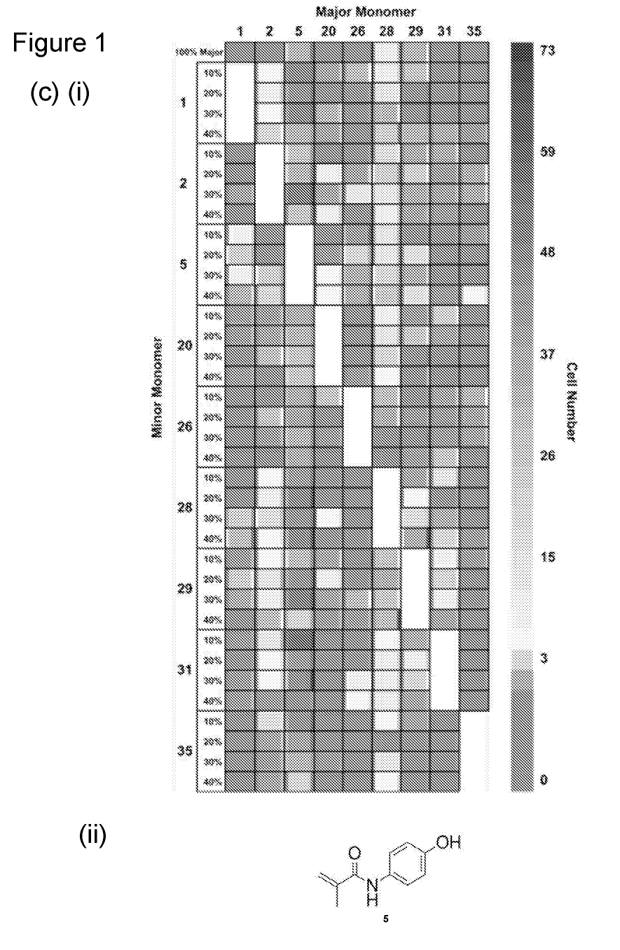

FIG. 1: Multi-generation array screening strategy. (a) (i) A first generation of wide chemically diversity consisting of 141 monomers was used to screen for hPSC attachment. (ii) Monomer identities of ‘hit’ materials identified from the first generation array screen, ‘hit’ materials were identified for the number of adhered cells after 24 hours of cell culture in StemPro medium (iii). (b) (i) 24 ‘hit’ materials were mixed pairwise in a combinatorial manner to produce a second-generation of 576 unique materials which were assessed and ranked in the similar way as the first-generation array. (ii) Monomer identities of ‘hit’ materials identified from the second generation array screen. (c) (i) A third-generation array was printed from 9 common monomers that formed the hit copolymers in the second-generation array but were mixed in further ratios to form an array of 297 materials. (ii) Materials were ranked to identify lead compositions for scale up, monomer 5 was found to be the lead candidate for scale up from the third generation array screening.

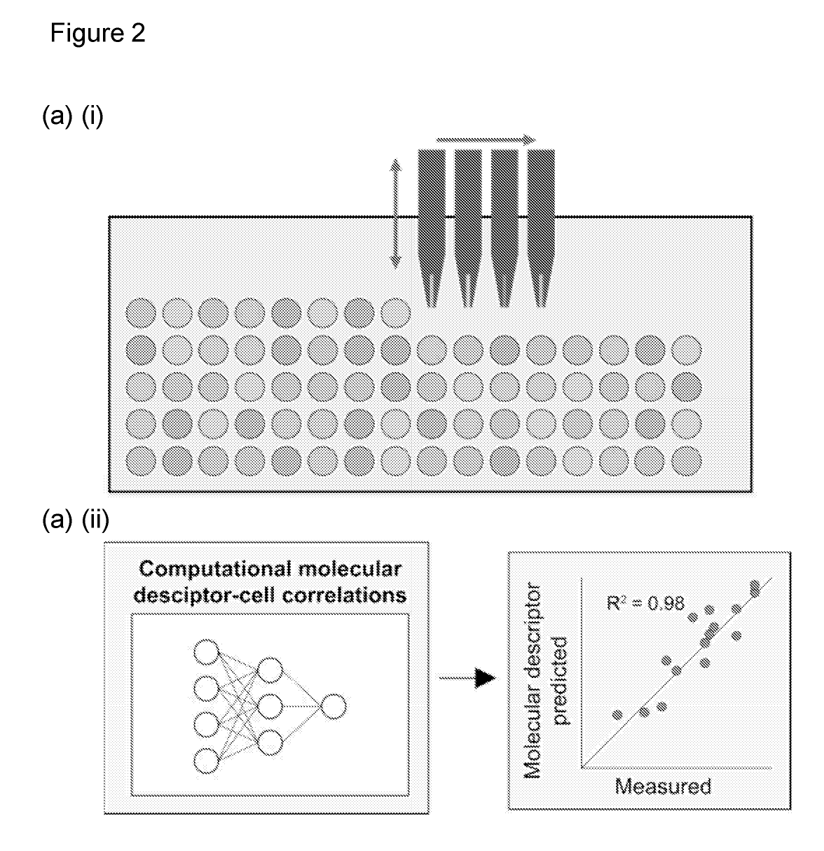

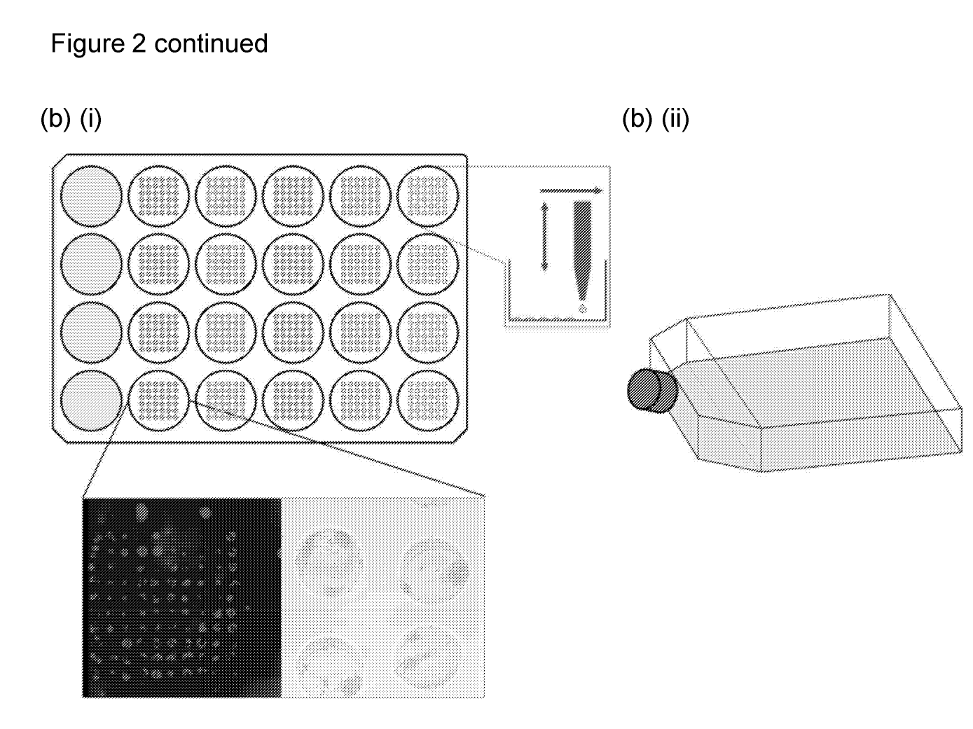

FIG. 2: Scalable polymeric materials for hPSC expansion. (a) (i) Contact printed polymer microarrays to screen for hPSC attachment and prediction of ‘hit’ materials using molecular descriptors (ii). (b) (i) Ink-jet printing of ‘hit’ materials identified from the multi-generation array screen into cultureware to assess hPSC attachment as an intermediate scale up format (ii). (c) Lead candidate scaled up to coat cultureware over large areas to assess long-term hPSC expansion potential.

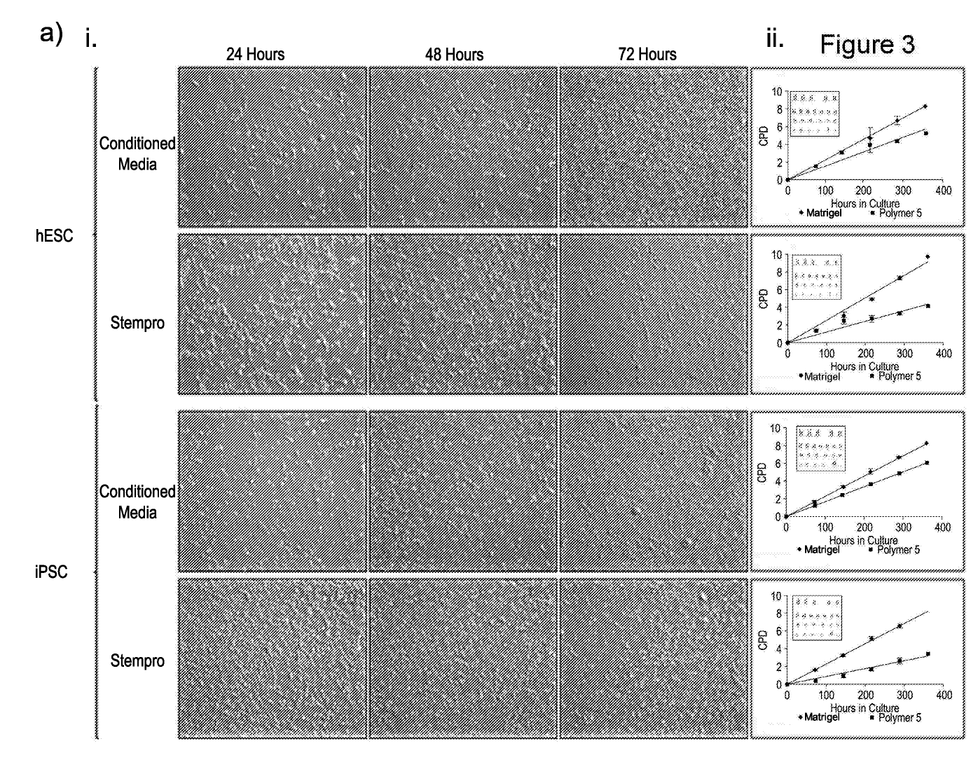

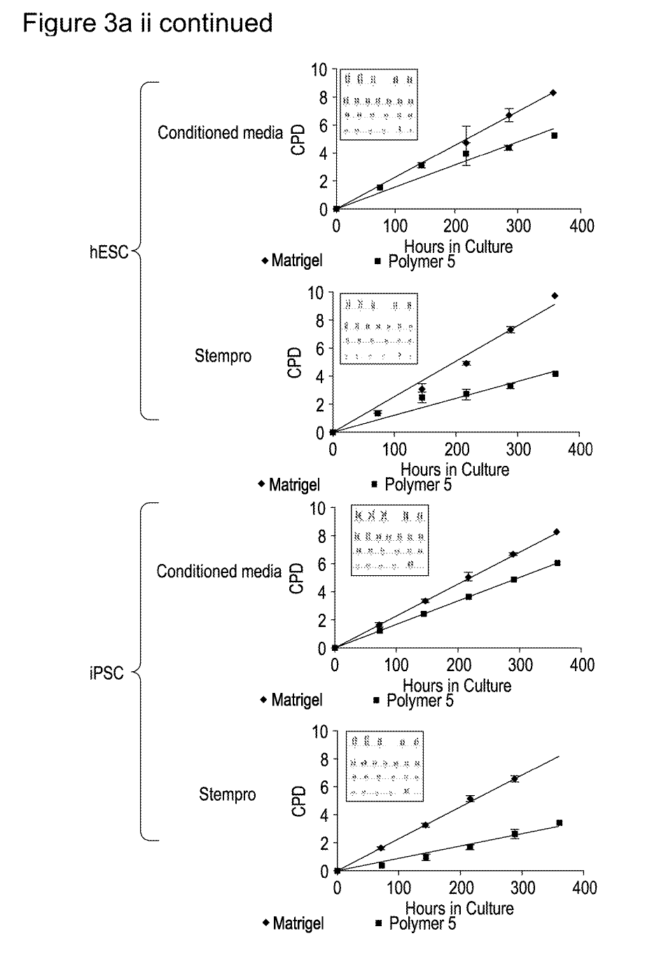

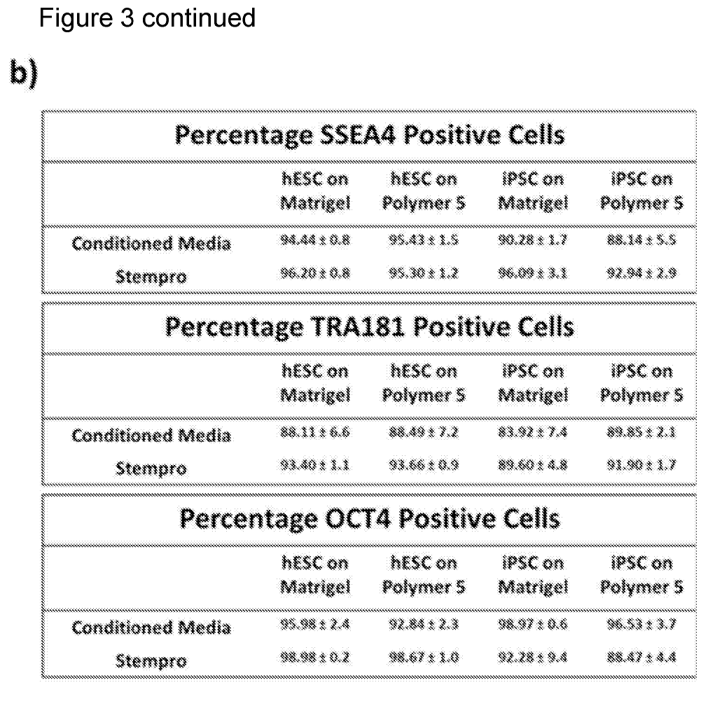

FIG. 3: Long-term hPSC expansion. (a) (i) hES and iPS cells were able to attach to Polymer 5 substrates at 24 h in and expand to confluence and compaction at 72 h. (ii) Growth curves showing doubling times of hPSCs on Polymer 5 versus Matrigel controls for 5 passages with maintenance of a normal karyotype. (b) Following long-term expansion on DM03 hPSCs maintain comparable pluripotent marker expression levels versus Matrigel controls.

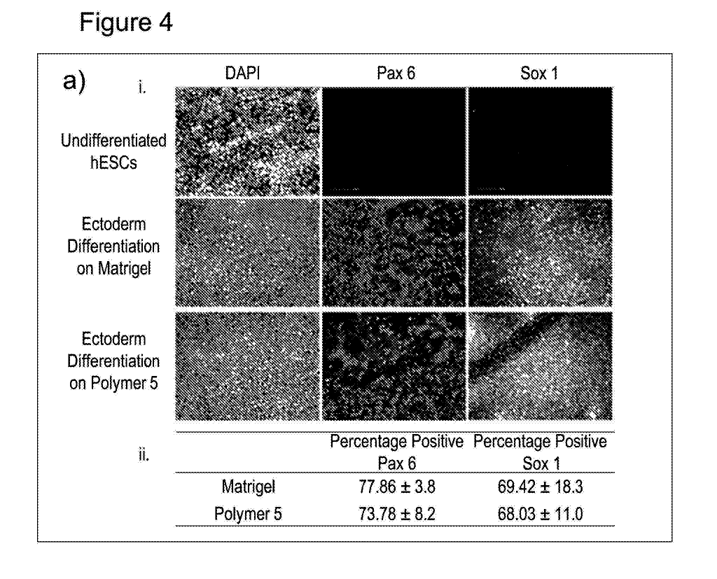

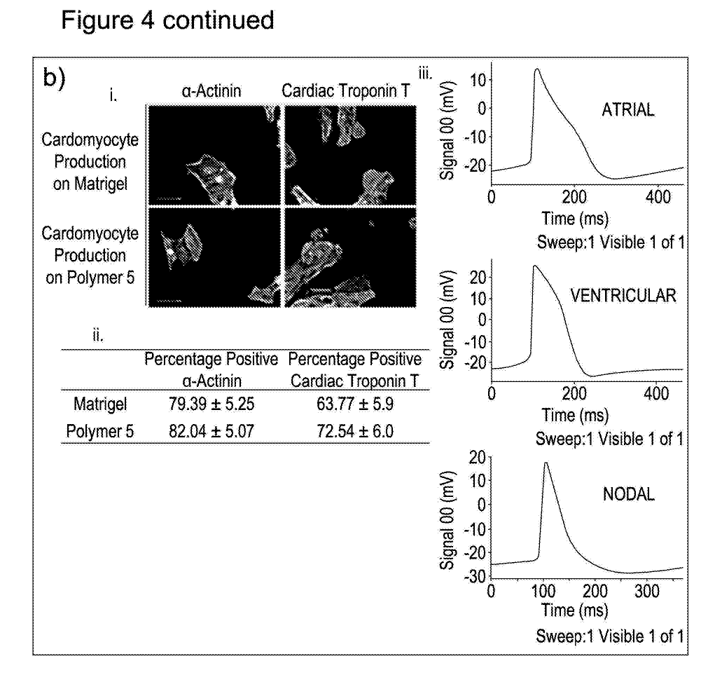

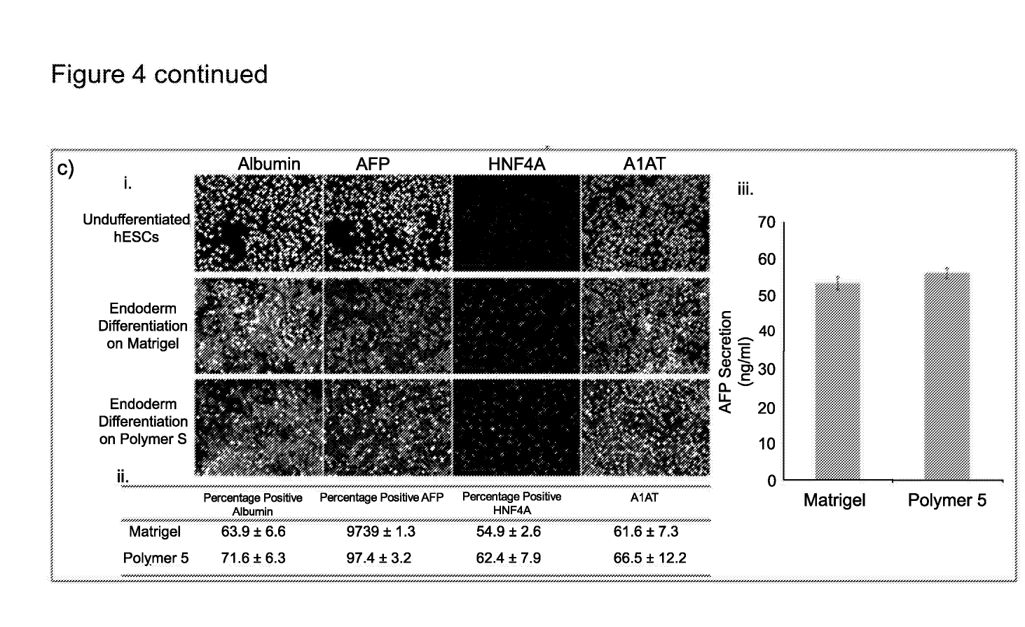

FIG. 4: Three germ layer differentiation of hPSCs on polymeric substrate. (a) (i) Ectoderm differentiation on Polymer 5 induced neurogenesis marker expression (ii). (b) (i) Mesoderm differentiation on Polymer 5 induced cardiac marker expression (ii) in spontaneously beating cardiomyocytes (iii). (c) (i) Endoderm differentiation on Polymer 5 induced hepatic marker expression (ii) in hepatocyte-like cells with active AFP secretion (iii).

### REFERENCE LIST OF MONOMERS BY NUMBER

Monomer 1—2-Norbornyl methacrylate
Monomer 2—2-Ethylhexyl methacrylate
Monomer 5—N-(4-Hydroxyphenyl)methacrylamide
Monomer 20—Octafluoro-2-hydroxy-6-(trifluoromethyl)heptyl methacrylate
Monomer 26—Lauryl methacrylate
Monomer 28—N-Phenylmethacrylamide
Monomer 29—Cyclohexyl methacrylate
Monomer 31—Dodecafluoroheptyl acrylate
Monomer 35—Tris[2-(acryloyloxy)ethyl] isocyanurate

The monomers have been numbered as above for ease of reference. Where reference is made to a polymer, the same numbering is intended to be used to denote that the polymer consists of the numbered monomer. For example, polymer 5 consists of monomer 5, polymer 1 would consist of monomer 1. A co-polymer of monomer 1 and monomer 5 could be named as poly-1-co-5, for example.

TABLE 1
Best performing materials from third generation array.
Cell number average (n = 5)

| Cell number | Monomer composition (% v/v) |
|---|---|
| 55.8 | 90% 5/10% 31 |
| 52.2 | 70% 5/30% 2 |
| 50 | 70% 5/30% 29 |
| 47.4 | 90% 5/10% 1 |
| 47.2 | 80% 5/20% 28 |
| 47 | 70% 5/30% 1 |
| 46.6 | 60% 5/40% 28 |
| 46 | 70% 5/30% 31 |
| 45 | 80% 5/20% 1 |
| 45 | 70% 5/30% 28 |
| 44.8 | 90% 5/10% 35 |
| 44.6 | 80% 5/20% 29 |
| 43 | 90% 5/10% 28 |
| 41.2 | 80% 5/20% 31 |
| 41 | 90% 26/10% 5 |
| 40.2 | 80% 5/20% 35 |
| 38.2 | 80% 5/20% 26 |
| 37.6 | 90% 5/10% 26 |
| 37.4 | 60% 5/40% 26 |
| 37.4 | 90% 5/10% 20 |
| 36.6 | 60% 26/40% 5 |
| 36.4 | 60% 29/40% 28 |
| 35.2 | 70% 26/30% 5 |
| 33.2 | 60% 5/40% 1 |
| 33 | 70% 5/30% 35 |
| 32.6 | 70% 5/30% 26 |
| 32.6 | 100% 5 |

Combinations and ratios in Table 1 are the result of a third generation array which is formed by mixing materials already known to be good for cell attachment and ranking these materials by their performance to choose the best materials for scaling up. This array consisted of 297 materials, the vast majority of which supported cell attachment. Table 1 highlights the excellent performance of the monomer 5 and its copolymers. To demonstrate this Table 1 lists the best performing materials until 100% of monomer 5 (homopolymer), this shows that copolymers of monomer 5 have a synergistic effect upon cell performance and display improved cell adhesion properties than monomer 5 alone.

Herein the invention provides a fully synthetic growth substrate for long-term hPSC culture in defined medium, which requires no preconditioning prior to cell culture. This polymeric material is amenable to scale up for automated hPSC expansion to achieve large numbers of cells that are necessary for clinical applications.

Results

To aid the discovery of new materials for hPSC culture, high-throughput screening sample formats such as polymer microarrays have been employed. A multi-generation array screening methodology was used to identify ‘hit’ materials that can support hPSC attachment over 24 h in defined StemPro medium (FIG. 1). The first generation array consisted of 141 monomers of wide chemical diversity including materials previously described to support the long-term hPSC culture. Irwin et al. (2011. Engineered polymer-media interfaces for the long-term self-renewal of human embryonic stem cells. Biomaterials. 32, 6912-6919) used a methacrylamide (N-(3-aminopropyl)methacrylamide hydrochloride that was grafted to the surface of TCPS dishes using a small quantity of crosslinker (N,N-methylenebis(acrylamide) and photoinitiator. Villa-Diaz et al. (2010. Synthetic polymer coatings for long-term growth of human embryonic stem cells. Nat. Biotech. 28, 581-583) grafted a methacrylate monomer to ozone-treated TCPS dishes by heating in an water/ethanol mixture at 80° C. From this initial screen, 24 ‘hit’ materials were chosen to form a second combinatorial array (70/30 v/v mixtures) of 576 unique materials. Hit materials were assessed by their ability to support OCT4+ cells after 24 h in culture. Second generation array hits were determined the same way, whereupon 9 hit monomers were identified and mixed in further ratios (10, 20, 30 and 40% v/v) in a combinatorial manner to produce a third generation array of 297 materials to further improved cell performance. Monomer 5 was selected from the third generation array to be scaled up into cultureware to assess hPSC expansion potential.

Monomer 5 was polymerized via a free radical polymerization in ethanol at 80° C. for 24 h. The polymer was isolated by precipitation into cold THF three times to remove excess monomer present. The dried polymer sample was redissolved into ethanol (5% w/v) and pipetted into oxygen plasma etched TCPS 6 well plates to cover the base of each well. Solutions were allowed to dry at ambient conditions for 24 hrs whereupon the coated cultureware is proceeded to cell culture.

This method of using a prepolymerised solution to coat cultureware is more convenient as large quantities can be synthesized in one batch that can be scaled up to industrial scale. Furthermore, the coating procedure is simple and can be performed routinely by hand or by a robotic fluid handling system to increase throughput of coated cultureware manufacture. The polymer may also be provided in powdered form for an end user to dissolve in solution and coat a cell culture surface.

Methods

Polymer Microarray Synthesis. Polymer microarrays were synthesised using methods previously described^{[1,21]}. Briefly, polymer microarrays were formed using a XYZ3200 dispensing station (Biodot) and metal pins (946MP3B, Arrayit). The printing conditions were 0 2<2000 ppm, 25° C., and 35% humidity. Polymerisation solution was composed of monomer (50% v/v) in dimethylformamide with photoinitiator 2,2-dimethoxy-2-phenyl acetophenone (1% w/v). Six replicates were printed on each slide. Monomers were purchased from Aldrich, Scientific Polymers and Polysciences and printed onto epoxy-coated slides (Xenopore) dip-coated with pHEMA (4% w/v, Sigma) in ethanol (95% v/v in water).

Preparation of Polymer-Coated Cultureware.

Micro-Array Screening

Top and bottom surfaces of array slides were sterilised by exposure to UV light for 15 minutes each. Slides were washed with sterile PBS three times and then exposed to StemPro® hESC SFM, a fully defined, serum- and feeder-free medium (Life Technologies, UK) for 1 hour at 37° C. with 5% CO2. The hESC line HUES7 (Cowan et al, (2004) N. Engl. J. Med., 350, 1353), was cultured on a Matrigel (BD Biosciences, UK) coated substrate in StemPro® containing 10 um Y-27632 (Tocris Bioscience, UK), for no higher than passage 35, prior to seeding on monomer arrays. Passaging of cells was achieved by incubation with accutase (Invitrogen, UK) for 3 min at 37° C., with tapping of the flasks to dissociate cells. 1.2×10⁶ HUES7 cells were seeded on each array and incubated at 37° C. with 5% CO2 for 24 hours to allow cell adhesion. Adherent cells were fixed in 4% paraformaldehyde (Sigma-Aldrich, UK) and permeabilised with 0.1% Triton-X 100 (Sigma-Aldrich, UK). Non-specific binding was blocked with 8% goat serum (Sigma-Aldrich, UK,) for 1 hour at room temperature. Samples were incubated with diluted mouse primary OCT4 antibody (1:200; Santa Cruz Biotech, Germany) overnight at room temperature. Cy3-conjugated goat anti-mouse IgG+IgM secondary antibody (1:250; Jackson Immuno Research, Inc., PA) was applied for 1 hour at room temperature. Samples were incubated with 4′,6-diamidino-2-phenylindole (DAPI) (1:1000; Sigma-Aldrich, UK,) for 10 minutes at room temperature and then mounted in Vectorshield mounting medium (Vector Labs, UK). Arrays were imaged using a fluorescence microscope (IMSTAR) and cell attachment determined using CellProfiler cell image analysis software (http://www.cellprofiler.org/).

Array Well Plates

Top and bottom surfaces of coated 24 well plates were sterilised by exposure to UV light for 15 minutes each, followed by washing with sterile PBS three times. 1.5×10⁵ HUES7 cells were seeded per well in StemPro® media containing 10 μm Y-27632 dihydrochloride and incubated at 37° C. with 5% CO2 for 24 hours to allow cell adhesion. Adherent cells were fixed in 4% paraformaldehyde (Sigma-Aldrich, UK) and permeabilised with 0.1% Triton-X 100 (Sigma-Aldrich, UK). Samples were incubated with 4′,6-diamidino-2-phenylindole (DAPI) (1:1000; Sigma-Aldrich, UK,) for 10 minutes at room temperature imaged using a Cellavista Plate Imaging Platform (Roche, UK).

Expansion Culture on Scaled up Surfaces

Top and bottom surfaces of coated 6 well plates were sterilised by exposure to UV light for 15 minutes each, followed by washing with sterile PBS three times. 6×10⁵ HUES7 cells were seeded per well in StemPro® media containing 10 μm Y-27632 dihydrochloride. Media was exchanged every 24 hours until cells reached confluence. After 72 hours cell passaging was achieved by incubation with accutase (Invitrogen, UK) for 3 min at 37° C., with tapping of the flasks to dissociate cells.

Time-of-Flight Secondary-Ion Mass Spectrometry

Measurements were conducted using a ToF-SIMS 4 (IONTOF GmbH) instrument operated using a 25 kV Bi3⁺ primary ion source exhibiting a pulsed target current of −1 pA. Samples were scanned at a pixel density of 100 pixels per mm, with 8 shots per pixel over a given area. An ion dose of 2.45×10¹¹ ions per cm² was applied to each sample area ensuring static conditions were maintained throughout. Both positive and negative secondary ion spectra were collected (mass resolution of >7000), over an acquisition period of fifteen scans (the data from which were added together). Owing to the non-conductive nature of the samples, charge compensation, in the form of a low energy (20 eV) electron floodgun, was applied.

### Expansion Culture on Scaled Up Surfaces

Top and bottom surfaces of coated 6 well plates were sterilised by exposure to UV light for 15 minutes each, followed by washing with sterile PBS three times. 6×10⁵ HUES? (hES) or BT1 (hiPS) cells were seeded per well in either MEF-Conditioned media or StemPro® media containing 10 μm Y-27632 dihydrochloride. Media was exchanged every 24 hours until cells reached confluence. After 72 hours cell passaging was achieved by incubation with accutase (Invitrogen, UK) for 3 min at 37° C., with tapping of the flasks to dissociate cells.

Ectoderm Differentiation

To induce ectoderm differentiation 2×10⁴ hPSCs were seed per well of polymer 5/pHEMA coated 96 well plates. Following 24 hours adhesion cells were subjected to daily media exchanges for seven days with 100 μl ectoderm inducing media that comprised of an advanced DMEM base media (Life Technologies), supplemented with 1× L-glutamine (Life Technologies), 1× CD Lipid Concentrate (Life Technologies), 7.5 ug/ml Transferrin (Sigma Aldrich), 14 ug/ml Insulin (Sigma Aldrich), 0.1 mM β-mercapto-ethanol, 10 uM SB431542 (Tocris), and 1 uM Dorsomorphin-1 (Tocris).

Endoderm Differentiation

1.7×10⁴ hPSCs were seed per well of polymer 5/pHEMA coated 96 well plates. Following 24 hours adhesion cells were subjected to daily media exchanges for three days (Day 1, 2 and 3) with 67 μl endoderm inducing medium A. Medium A comprised a RPMI base medium (Life Technologies) supplemented with 1× B-27 (Life Technologies), 100 ng/ml Activin A (Life Technologies), and 50 ng/ml Wnt3a (RnD). On days 4, 6 and 8 media exchanges were perform with 134 μl endoderm inducing medium B. Medium B comprised a DMEM/F12 base media (Life Technologies), supplemented with 15% KSR (Life Technologies), 1% NEAA (Life Technologies), 1% Glutamax, 0.01% β-mercaptoethanol (Sigma), 4 ng/ml bFGF (Peprotech), and 1% DMSO (Sigma). On days 9, 11 and 13 media exchanges were perform with 13411 endoderm inducing medium C. Medium C comprised Leibowitz L15 base medium (Sigma), supplemented with 8.3% Tryptose phosphate broth (Sigma), 8.3% heat inactivated FBS (Sigma), 1 μM Insulin (Sigma), 10 μM Hydrocortisone (Sigma), 0.83% L-Glutamine (Life Technologies), 0.245 μM Ascorbic Acid (Sigma), 10 ng/ml HGF (Peprotech), and 20 ng/ml Oncostatin-M (RnD). Final media exchanges of 34 μl endoderm inducing Medium C were performed on days 15, 16 and 17.

Mesoderm Differentiation

2.5×10⁴ hPSCs were seed per well of polymer 5/pHEMA coated 96 well plates and allowed to expanded for 72 hours to reach confluence. At this time point (day 1) differentiation was initiated by exposure to 100 μl mesoderm inducing medium A. Medium A comprised Stempro34 medium (Life Technologies), supplemented 8 ng/ml Activin A (Life Technologies), and 10 ng/ml BMP4 (RnD). Media exchanges were performed on days 3 and 5 with 100 μl mesoderm inducing medium B. Medium B comprised a RPMI base medium (Life Technologies), supplemented with 1× B-27 (Life Technologies), 10 μM KY02111 (RnD) and 10 μM XAV939 (RnD). From day 7 onwards media exchanges were performed every other day with RPMI base medium (Life Technologies) supplemented with 1× B-27 (Life Technologies).

### REFERENCES

1 D. G. Anderson, S. Levenburg and R. Langer, Nat. Biotech., (2004), 22, 863.
2. Place, E. S., Evans, N. D. & Stevens M. M. Complexity in biomaterials for tissue engineering. Nature Mater. 8, 457-470 (2009).
3. Hook, A. L. et al. High throughput methods applied in biomaterials development and discovery. Biomaterials 31, 187-198 (2010).
11. Martin, M. J., Muotri, A., Gage, F. & Varki, A. Human embryonic stem cells express an immunogenic nonhuman sialic acid. Nature Med. 11, 228-232 (2005).
12. Kleinman, H. K. et al. Isolation and characterization of type-IV procollagen, laminin, and heparin-sulfate proteoglycans from EHS sarcoma. Biochem. 21, 6188-6193 (1982).
13. Jin, S., Yao, H., Weber, J. L., Melkoumian, Z. K. & Ye, K. A synthetic, xeno-free peptide surface for expansion and directed differentiation of human induced pluripotent stem cells. PLOS ONE 7:11 (2012).
14. Nagaoka, M., Si-Tayeb, K., Akaike, T. & Duncan, S. A. Culture of human pluripotent stem cells using completely defined conditions on a recombinant E-cadherin substratum. BMC Dev. Bio. 10:60 (2010).
16. Swistowski, A. et al. Xeno-free defined conditions for culture of human embryonic stem cells, neural stem cells and dopaminergic neurons derived from them. PLOS ONE 4:7 (2009)
17. Ludwig, T. E. et al. Derivation of human embryonic stem cells in defined conditions. Nature Biotech. 24, 185-187 (2006).
18. Wang, L. et al. Self-renewal of human embryonic stem cells requires insulin-like growth factor-1 receptor and ERBB2 receptor signalling. Blood 110, 4111-4119 (2006).
19. Bergstrom, R., Strom, S., Holm, F., Feki, A. & Hovatta, O. Xeno-free culture of human pluripotent stem cells. Methods Mol. Biol. 767, 125-136 (2011).
20. Chen, G. et al. Chemically defined conditions for human iPSC derivation and culture. Nature Meth. 8, 424-429 (2011).
21. A. L. Hook, C. Chien-Yi, J. Yang, D. J. Scurr, R. Langer, D. G. Anderson, S. Atkinson, P. Williams, M. C. Davies and M. R. Alexander, J. Visualized Exp., (2012), DOI:10.3791/3636.

1. A substrate for cell culture comprising a polymer, wherein the polymer comprises
a homopolymer formed from a monomer of formula (Ia) or (Ib);
a copolymer formed from one or more monomers of formula (Ia) and/or (Ib); or
a copolymer formed from one or more monomers of formula (Ia) and/or (Ib), and comprising HEMA;
wherein formula (Ia) comprises: 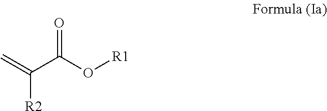
wherein
R1 is a C8-C12 straight or branched chain alkyl or alkenyl group, for example a C8-C10 straight or branched chain alkyl group, which may optionally be substituted; and
R2 is selected from H and C1-4 alkyl; and
wherein formula (Ib) comprises: 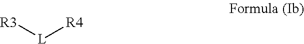
wherein
R3 is a 6-12 membered ring, for example a 6-8 membered ring, which is a cycloalkyl, cycloheteroalkyl, aryl or heteroaryl group, and which may optionally be substituted;
L is a divalent linker group selected from —NH—, —CH₂—, and —O—;
R4 is a C1-8 organic group, for example a C1-C6 organic group, wherein this group comprises at least one moiety selected from C═O, NH, NH₂, COOH, and C═C.

2. The Substrate according to claim 1, wherein R2 is selected from H and C1 or C2 alkyl.

3. The substrate according to claim 1 or claim 2, wherein the R1 group and/or the R3 group is substituted.

4. The substrate according to any preceding claim, wherein R3 is a cycloheteroalkyl, or heteroaryl group, and wherein the heteroatoms in the ring are selected from O, N, S, SO₂, P, B, Si, and combinations thereof; or R3 is cycloalkyl or aryl with no heteroatoms in the ring.

5. The substrate according to any preceding claim, wherein R3 is a 6 membered ring.

6. The substrate according to any preceding claim, wherein the R4 group is straight chain or branched.

7. The substrate according to any preceding claim, wherein the R4 group is a C1-C5 organic group, such as a C1-C4 organic group.

8. The substrate according to any preceding claim, wherein the R4 organic group comprises two or more moieties selected from C═O, NH, NH₂, COOH, and C═C.

9. The substrate according to any preceding claim, wherein the R4 group is 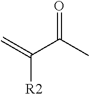
where R2 is selected from H and C1-4 alkyl; preferably it is selected from H and C1 or C2 alkyl, for example it may be H or C1 alkyl.

10. The substrate according to any preceding claim, wherein the monomer of formula (Ia) comprises any of the monomers selected from:
2-Ethylhexyl methacrylate: 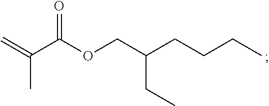
Octafluoro-2-hydroxy-6-(trifluoromethyl)heptyl methacrylate: 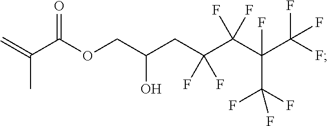 [SMILES: C=C(C)C(=O)OCC(O)CC(F)(F)C(F)(F)C(F)(C(F)(F)F)C(F)(F)F]
Dodecafluoroheptyl acrylate: 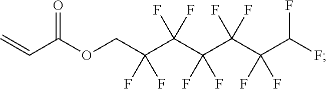 [SMILES: C=CC(=O)OCC(F)(F)C(F)(F)C(F)(F)C(F)(F)C(F)(F)C(F)F]
or
Lauryl methacrylate:

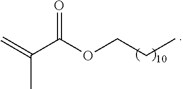

11. The substrate according to any preceding claim, wherein the monomer of formula (Ib) comprises any of the monomers selected from:
N-(4-Hydroxyphenyl)methacrylamide: 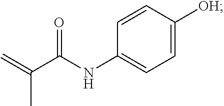
2-Norbornyl methacrylate: 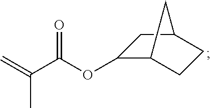
N-Phenylmethacrylamide: 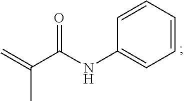
Cyclohexyl methacrylate: 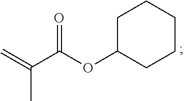
Tris[2-(acryloyloxy)ethyl] isocyanurate: 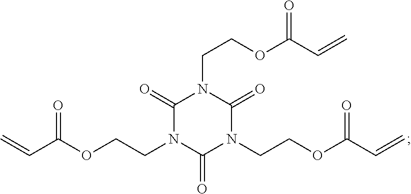 [SMILES: C=CC(=O)OCCn1c(=O)n(CCOC(=O)C=C)c(=O)n(CCOC(=O)C=C)c1=O]
Poly-L-Tyrosine: 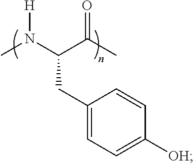
or
Dopamine:

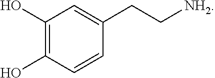 [SMILES: NCCc1ccc(O)c(O)c1]

12. The substrate according to any preceding claim, wherein the monomer of formula (Ib) comprises any of the monomers selected from N-(4-Hydroxyphenyl)methacrylamide; 2-Norbornyl methacrylate; N-Phenylmethacrylamide; Cyclohexyl methacrylate; or Tris[2-(acryloyloxy)ethyl] isocyanurate; or
wherein the monomer of formula (Ib) comprises Poly-L-Tyrosine; or
wherein the monomer of formula (Ib) comprises Dopamine.

13. The substrate according to any preceding claim, wherein the substrate comprises a polymer blend of two or more different polymers.

14. The substrate according to claim 13, wherein the second and/or third polymers comprise a homopolymer formed from a monomer of formula (Ia) or (Ib); or a copolymer formed from one or more monomers of formula (Ia) and/or I(b); or a copolymer formed from one or more monomers of formula (Ia) and/or (Ib) and comprising HEMA.

15. The substrate according to any preceding claim, wherein the polymer comprises a copolymer, and the copolymer comprises at least 10% HEMA.

16. The substrate according to any preceding claim, wherein the copolymer comprises N-(4-Hydroxyphenyl)methacrylamide and HEMA.

17. The substrate according to any preceding claim, wherein distinct regions of polymers comprise arrangements of strips, spots, lattices, or layers of one type of polymer alongside distinct regions or layers of another polymer.

18. The substrate according to any preceding claim, wherein the substrate for cell culture is a coating on the surface of a cell culture device.

19. The substrate according to any of claims 1 to 17, wherein the substrate is in a solution, arranged to be applied to a surface of a cell culture device.

20. The substrate according to any of claims 1 to 17, wherein the substrate is in powdered form, wherein the substrate is arranged to be dissolved in solution and applied to a cell culture device.

21. The substrate according to any preceding claim, wherein the cell culture device comprises a tissue culture plate.

22. The substrate according to any preceding claim, wherein the cells to be cultured with or on the substrate comprise or consist of stem cells; partially or fully differentiated cells, such as fibroblasts; or progenitor cells; or combinations thereof.

23. The substrate according to any preceding claim, wherein the cells comprise or consist of pluripotent stem cells or multipotent stem cells.

24. The substrate according to any preceding claim, wherein the cells comprise or consist of human pluripotent stem cells (hPSCs) or human mesenchymal stem cells (hMSCs).

25. A cell culture device comprising the substrate for culturing cells in accordance with any preceding claim.

26. A polymer for use as a substrate for facilitating cell adhesion on a cell culture plate; wherein the polymer comprises
a homopolymer formed from a monomer of formula (Ia) or (Ib);
a copolymer formed from one or more monomers of formula (Ia) and/or (Ib); or
a copolymer formed from one or more monomers of formula (Ia) and/or I(b), and comprising HEMA.

27. Use of the polymer according to claim 26 as a substrate for facilitating cell adhesion on a cell culture plate.

28. A method of culturing cells, the method comprising the steps of:
providing a device for cell culture, wherein the device for cell culture comprises a surface layered with the substrate in accordance with any of claims 1 to 24;
introducing cell culture media and the cells; and
incubating the device at a temperature suitable for maintenance and/or growth of the cells.

29. The method according to claim 28, further comprise the step of harvesting cultured cells from the device, wherein the cells are harvested by enzymatic removal from the surface of the device.

30. The method according to claim 28 or 29, wherein the cell culture is for at least 24 hours.

31. The method according to any of claims 28 to 30, wherein the method comprises one or more passages for the period of culture.

32. The method according to any of claims 28 to 31, wherein the cell culture comprises hPSCs maintained for at least 2 passages.

33. The method according to any of claims 28 to 32, wherein the cell culture comprises hPSCs maintained for at least 4 days.

34. The method according to any of claims 28 to 33, wherein pluripotent stem cells are cultured, such as hPSCs, and the cell culture maintains pluripotency for at least 12 hours.

35. The method according to any of claims 28 to 34, wherein the method of culturing cells comprises growing or maintaining stem cells, such as hPSCs, on the substrate, followed by removing the cells from the substrate and differentiating the cells.

36. The method according to any of claims 28 to 34, wherein the the method of culturing cells comprises growing or maintaining stem cells, such as hPSCs, on the substrate, followed by differentiating the cells on the substrate.

37. A method of maintaining pluripotency of hPSCs or multipotency of a MSC cell in culture in vitro, comprising culturing the hPSCs on a substrate in accordance with any of claims 1 to 24.

38. A method of maintaining differentiation of a differentiated cell in a culture in vitro, comprising culturing the differentiated cell on a substrate in accordance with any of claims 1 to 24.

39. A method of promoting differentiation of a stem cell in a culture in vitro, comprising culturing the stem cell on a substrate in accordance with any of claims 1 to 24.

40. A method of maintaining a stem cell culture in vitro during the promotion of differentiation of the stem cells, the method comprising culturing the stem cell on a substrate in accordance with any of claims 1 to 24.

41. Use of the device according to claim 25 for culturing cells, optionally wherein the cells comprise human pluripotent stem cells.

42. A method of manufacturing a cell culture plate for culturing cells, wherein the cell culture plate is provided with coating of the substrate according to any of claims 1 to 24.

43. The method according to claim 42, wherein the substrate is manufactured by polymer self-assembly, or copolymer self-assembly; and optionally wherein the substrate is manufactured by uncontrolled free radical polymerisation of polymers or copolymers.

44. The method according to claim 42 or claim 43, wherein the substrate is coated on the cell culture device by printing.

45. The method according to any of claims 42 to 44, wherein the substrate is coated on the cell culture device by applying a solution of the polymer in a solvent to the cell culture device, and allowing or causing evaporation of the solvent.

46. A method of manufacturing stem cells, comprising culturing stem cells in the device in accordance with claim 25, or culturing cells according to the method of any of claims 28 to 40.

47. A substrate; use; polymer; device; or method, substantially as described herein, optionally with reference to the accompanying drawings.
